(19) United States
(12) Patent Application Publication
(10) Pub. No.: US 2025/0135170 A1
(43) Pub. Date: May 1, 2025

(54) COMPOSITIONS AND ARTICLES FOR DELIVERING NITRIC OXIDE AND METHODS FOR MAKING AND USING THE SAME

(71) Applicant: University of Georgia Research Foundation, Inc., Athens, GA (US)

(72) Inventors: Manjyot Kaur Chug, Athens, GA (US); Elizabeth Brisbois, Athens, GA (US); Hitesh Handa, Athens, GA (US); Natalie Crutchfield, Athens, GA (US)

(21) Appl. No.: 18/923,765

(22) Filed: Oct. 23, 2024

Related U.S. Application Data
(60) Provisional application No. 63/593,307, filed on Oct. 26, 2023.

(51) Int. Cl. A61M 31/00; A61K 33/00; A61K 47/34; A61P 1/02

(52) U.S. Cl. CPC A61M 31/002; A61K 33/00; A61K 47/34; A61P 1/02

(57) ABSTRACT

Described herein are compositions and articles for delivering nitric oxide to a subject. The compositions described herein can eradicate pathogens in a subject. In one aspect, the compositions described herein can be applied as a coating to a medical device. In other aspects, the compositions described herein can be used to produce a medical device that can release nitric oxide. The compositions and articles described herein can be used to treat or prevent bacterial infections and biofilm formation in a subject.

### CROSS-REFERENCE TO RELATED APPLICATIONS

This application claims the benefit of and priority to co-pending U.S. Provisional Patent Application No. 63/593,307, filed on Oct. 26, 2023, the contents of which are incorporated by reference herein in their entireties.

### STATEMENT REGARDING FEDERALLY SPONSORED RESEARCH OR DEVELOPMENT

This invention was made with government support under grant number R01 HL157587 awarded by the NIH. The government has certain rights in the invention. (37 CFR 401.14 f (4)”

### BACKGROUND

Severe periodontal diseases affect more than 19% of the global population, with over 40% of adults in the United States being affected by periodontitis despite a majority of these oral health conditions being readily preventable [1, 2]. Periodontal disease is characterized by aggregation of bacteria in the periodontal pocket within the gums that begins as gingivitis and progresses to periodontitis. Once advanced to periodontitis, there is significant soft tissue damage and alveolar bone loss [3]. Severe periodontitis is one of the world's most prevalent inflammatory diseases. There is a predictable increase in the healthcare burden of periodontal disease as the population and life expectancy are increasing [4]. Considering the indirect costs, in 2018, periodontal disease caused an estimated loss of $154.06B in the US and €158.64B in Europe [5]. The current point of care for periodontitis includes scaling, root planning, and antibiotics in the form of mouthwashes and gels, which have limited reach to the periodontal pockets, and micro-periodontal surgery [2]. Treatment for periodontitis requires clinical intervention, leading to significant economic costs and healthcare disparities. Therefore, creating an affordable prevention method for periodontal disease, such as bioactive coatings for interdental cleaning (i.e., dental floss) is of interest for at-home care. Such technologies have the potential to improve patient accessibility and compliance, improving general health outcomes.

### SUMMARY

Described herein are compositions and articles for delivering nitric oxide to a subject. The compositions described herein can eradicate pathogens in a subject. In one aspect, the compositions described herein can be applied as a coating to a medical device. In other aspects, the compositions described herein can be used to produce a medical device that can release nitric oxide. The compositions and articles described herein can be used to treat or prevent bacterial infections and biofilm formation in a subject.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIGS. 1A-1C show a schematic representation of NO releasing dental floss for an advanced care of the subgingival region. (A)NO-releasing floss is fabricated using a SNAP-PEG mixture coating. Various amounts of SNAP can be loaded into the mixture to enhance the tunability of NO release on surface. (B) The versatility of SNAP-PEG mixture is shown as solutions when heated to 60° C. and as a solid substrate at room temperature (23° C.). (C) Delivery of drugs using SNAP-PEG coated dental floss involves a gradual transfer of SNAP from the floss onto the surfaces of teeth and the gingiva within the subgingival region during the flossing process. This results in the deposition of the coating into the periodontal pocket, enabling a sustained release of NO over an extended period.

FIGS. 2A-2D show (A) UV-vis spectroscopy absorption data was used to determine the concentration of each weight percent formulation incorporated (N=5). (B) Determination of uniformity of the SNAP-PEG coated NO-releasing floss to illustrate the uniformity in the fabrication (N=16). An indirect drug efficiency study (C) Drug delivery efficiency of SNAP-PEG coated floss in depositing the coating between the teeth of a tooth model. (D) SEM Images shows the morphology of the 1 wt % SNAP-PEG coating on floss before and after deposition of the coating in a tooth model. The scale bars represent 500 μm.

FIGS. 3A-3D show (A) representative instantaneous NO release profiles of each weight percent of SNAP-PEG coating over a 30 h period. (B) Average NO flux recorded from 1,5 and 10 wt % of SNAP-PEG coated dental floss at varying time points (0, 2, 6, 30 h) reported as the mean±SD determined using nitric oxide analyzer (N=3). (C) The total nitrite concentration was used to estimate the NO-release in aqueous solution reported as the mean±SD (N=4). (D) Storage stability analysis of NO-releasing SNAP-PEG coated floss over 28 d at room temperature (N=3).

FIGS. 4A-4D show in vitro evaluation of NO-releasing SNAP-PEG coatings. (A)NO-releasing dental floss with inherent broad-spectrum antimicrobial and anti-inflammatory properties can help in the prevention of periodontal infections. (B) Zone of inhibition studies were performed with S. mutans and E. coli. Final data are shown as mean±SD (N=5). Statistical significance is presented as *** (p<. 001), and **** (p<. 0001). (C) Representational images of zone of inhibition on agar plates for S. mutans and E. coli with the labels I-IV being the PEG control, 1 wt %, 5 wt %, and 10 wt % respectively (D). Relative cell viability towards hFOB 1.19 and HGF cell lines. Data is presented as the mean percent viability normalized against untreated cells±SD (N=3).

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

Before the present compounds, compositions, articles, devices, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions and Abbreviations

In describing and claiming the disclosed subject matter, the following terminology will be used in accordance with the definitions set forth below.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a solvent” includes, but are not limited to, mixtures or combinations of two or more such solvents, and the like.

It should be noted that ratios, concentrations, amounts, rates, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed and “about 5 to about 15” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance and instances where it does not.

As used herein, the term “biocompatible,” with respect to a substance or fluid described herein, indicates that the substance or fluid does not adversely affect the short-term viability or long-term proliferation of a target biological particle within a particular time range.

The terms “antimicrobial” and “antimicrobial characteristic” refer to the ability to kill and/or inhibit the growth of microorganisms. A substance having an antimicrobial characteristic may be harmful to microorganisms (e.g., bacteria, fungi, virus, protozoans, algae, and the like). A substance having an antimicrobial characteristic can kill the microorganism and/or prevent or substantially prevent the growth or reproduction of the microorganism.

The terms “bacteria” or “bacterium” include, but are not limited to, gram positive and gram negative bacteria. Bacteria can include, but are not limited to, Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anabaena affinis and other cyanobacteria (including the Anabaena, Anabaenopsis, Aphanizomenon, Camesiphon, Cylindrospermopsis, Gloeobacter Hapalosiphon, Lyngbya, Microcystis, Nodularia, Nostoc, Phormidium, Planktothrix, Pseudoanabaena, Schizothrix, Spirulina, Trichodesmium, and Umezakia genera) Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Phytoplasma, Plesiomonas, Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Spiroplasma, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella. Other examples of bacterium include Mycobacterium tuberculosis, M. bovis, M. typhimurium, M. bovis strain BCG, BCG substrains, M. avium, M. intracellulare, M. africanum, M. kansasii, M. marinum, M. ulcerans, M. avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthracis, B. subtilis, Nocardia asteroides, and other Nocardia species, Streptococcus viridans group, Peptococcus species, Peptostreptococcus species, Actinomyces israelii and other Actinomyces species, and Propionibacterium acnes, Clostridium tetani, Clostridium botulinum, other Clostridium species, Pseudomonas aeruginosa, other Pseudomonas species, Campylobacter species, Vibrio cholera, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species Brucella abortus, other Brucella species, Chlamydi trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteria, Brucella abortus and other Brucella species, Burkholderia cepacia, Burkholderia pseudomallei, Francisella tularensis, Bacteroides fragilis, Fudobascterium nucleatum, Provetella species, and Cowdria ruminantium, or any strain or variant thereof. The gram-positive bacteria may include, but is not limited to, gram positive Cocci (e.g., Streptococcus, Staphylococcus, and Enterococcus). The gram-negative bacteria may include, but is not limited to, gram negative rods (e.g., Bacteroidaceae, Enterobacteriaceae, Vibrionaceae, Pasteurellae and Pseudomonadaceae).

The term “antimicrobial effective amount” as used herein refers to that amount of the compound being administered/released that will kill microorganisms or inhibit growth and/or reproduction thereof to some extent (e.g. from about 5% to about 100%). In reference to the compositions or articles of the disclosure, an antimicrobial effective amount refers to that amount which has the effect of diminishment of the presence of existing microorganisms, stabilization (e.g., not increasing) of the number of microorganisms present, preventing the presence of additional microorganisms, delaying or slowing of the reproduction of microorganisms, and combinations thereof. Similarly, the term “antibacterial effective amount” refers to that amount of a compound being administered/released that will kill bacterial organisms or inhibit growth and/or reproduction thereof to some extent (e.g., from about 5% to about 100%). In reference to the compositions or articles of the disclosure, an antibacterial effective amount refers to that amount which has the effect of diminishment of the presence of existing bacteria, stabilization (e.g., not increasing) of the number of bacteria present, preventing the presence of additional bacteria, delaying or slowing of the reproduction of bacteria, and combinations thereof.

As used herein, the term “subject” includes humans, mammals (e.g., cats, dogs, horses, etc.), birds, and the like. Typical subjects to which embodiments of the present disclosure may be administered will be mammals, particularly primates, especially humans. For veterinary applications, a wide variety of subjects will be suitable, e.g., livestock such as cattle, sheep, goats, cows, swine, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like.

The terms “treat”, “treating”, and “treatment” are an approach for obtaining beneficial or desired clinical results. Specifically, beneficial or desired clinical results include, but are not limited to, alleviation of symptoms, diminishment of extent of disease, stabilization (e.g., not worsening) of disease, delaying or slowing of disease progression, substantially preventing spread of disease, amelioration or palliation of the disease state, and remission (partial or total) whether detectable or undetectable.

The term “oral cavity” is the mouth of the subject and includes the teeth, hard late, soft plate, uvula, tonsils, floor of the mouth, tongue, inferior labial frenulum, retromolar trigone, palatine arch, glossopalatine arch, gingiva, and superior labial frenulum.

The term “periodontal disease” also known as gum disease, is a set of inflammatory conditions affecting the tissues surrounding the teeth. In its early stage, called gingivitis, the gums become swollen, red, and may bleed. It is a cause of tooth loss for adults. In its more serious form, called periodontitis, the gums can pull away from the tooth, bone can be lost, and the teeth may loosen or fall out.

### Compositions

In accordance with the purpose(s) of the present disclosure, described herein are compositions and articles for delivering nitric oxide to a subject. The compositions described herein can eradicate pathogens in a subject. In one aspect, the compositions described herein can be applied as a coating to a medical device. In other aspects, the compositions described herein can be used to produce a medical device that can release nitric oxide. The compositions and articles described herein can be used to treat or prevent bacterial infections and biofilm formation in a subject.

In one aspect, the compositions described herein include a mixture composed of a polymer and a nitric oxide releasing compound. Methods for preparing and using the compositions described is described in detail below.

### Polymers

The polymers described herein are biocompatible and are soluble in a variety of solvents including water. The solubility of the polymers permits easy mixing of the nitric oxide releasing compound in the polymer at varying amounts. In one aspect, the polymer is a hydrophilic polymer. The polymers are biocompatible and biodegradable so that the nitric oxide releasing compound can be released from the composition over time.

In one aspect, the polymer is a polyacrylamide, polybetaine, a poloxamer, a polyester, a polypeptoid (e.g., polysarcosine), a polyalkylene glycol (e.g., polyethylene glycol), a polyalkylene (e.g., polypropylene), polylactic acid, polyglycolic acid, poly-d, I-lactic-co-glycolic acid (PLGA), glycerin, poly-N-vinylpyrrolidone, or any combination thereof.

In one aspect, the polymer can include two or more different polymers. In another aspect, the polymer can include two or more polymers having the same chemical structure, where each polymer has a different molecular weight. In one aspect, the polymer has an average molecular weight of from about 500 Da to about 10,000 Da.

In one aspect, the polymer comprises polyethylene glycol. In one aspect, polyethylene glycol has an average molecular weight of from about 500 Da to about 10,000 Da, or about 500 Da, 1,000 Da, 2,000 Da, 3,000 Da, 4,000 Da, 5,000 Da, 6,000 Da, 7,000 Da, 8,000 Da, 9,000 Da, or 10,000 Da, where any value can be a lower and upper endpoint of a range (e.g., 2,000 Da to 5,000 Da).

In one aspect, the polymer includes polyethylene glycol with a first polyethylene glycol and a second polyethylene glycol, wherein the first polyethylene glycol and the second polyethylene glycol have a different average molecular weight. In one aspect, the first polyethylene glycol has an average molecular weight of from about 500 Da to about 2,000 Da, or about 500 Da, 750 Da, 1,000 Da, 1,250 Da, 1,500 Da, 1,750 Da, or 2,000 Da, where any value can be a lower and upper endpoint of a range (e.g., 750 Da to 1,250 Da). In another aspect, the second polyethylene glycol has an average molecular weight of from about 2,500 Da to about 4,000 Da, or about 2,500 Da, 2,750 Da, 3,000 Da, 3,250 Da, 3,500 Da, 3,750 Da, or 4,000 Da, where any value can be a lower and upper endpoint of a range (e.g., 3,000 Da to 3,750 Da).

The weight ratio of the first and second polyethylene glycol can vary. In one aspect, the weight ratio of the first polyethylene glycol to the second first polyethylene glycol is from 1:3 to 3:1, or about 3:1, 2.5:1, 2:1, 1.5:1, 1:1, 1:1.5, 1:2.0, 1:2.5, or 1:3, where any value can be a lower and upper endpoint of a range (e.g., 1:1.5 to 1:2.5).

In one aspect, when the polymer is polyethylene glycol (i.e., one or more polymers of polyethylene glycol), the polyethylene glycol has a viscosity of about 50 centipoise to about 500 centipoise, or about 50 centipoise, 100 centipoise, 150 centipoise, 200 centipoise, 250 centipoise, 300 centipoise, 350 centipoise, 400 centipoise, 450 centipoise, or 500 centipoise, where any value can be a lower and upper endpoint of a range (e.g., 150 centipoise to 250 centipoise).

In another aspect, the polymer can include a polymer with polyethylene glycol subunits or a polymer functionalized with polyethylene glycol. In one aspect, polyethylene glycol subunits can be incorporated into the polymer backbone or attached to the polymer as side chains. In one aspect, the polymer is a polyurethane functionalized with polyethylene glycol subunits.

### Nitric Oxide Releasing Compounds

The nitric oxide releasing compound is a compound that possesses one or more nitric oxide groups, wherein nitric oxide is subsequently released from the compound. In one aspect, the nitric oxide releasing compound is an S-nitrosothiol compound. In another aspect, the nitric oxide compound is S-nitroso-N-acetyl-penicillamine, S-nitroso-N-acetylcysteine, S-nitroso-N-acetyl cysteamine, S-nitrosoglutathione, S-nitrosocysteamine-glutathione, methyl S-nitrosothioglycolate, nitrosated cysteine, S-nitroso-N-acetyl-I-cysteine ethyl ester (SNACET), S-nitroso-L-homocysteine, S-nitroso-L-cysteine, S-nitroso-albumin, S-nitrosocaptopril, or any combination thereof, or any combination thereof.

In another aspect, the nitric oxide releasing compound can be S-nitrosothiol conjugated polymers, modified-dendrimers; S-nitrosothiol modified polysaccharides, S-nitrosothiol modified nano/microparticles, or S-nitrosothiol modified-proteins. In another aspect, the nitric oxide releasing compound can include NO-donors such as, for example, nitrates, N-diazeniumdiolates (NONOates), and S-nitrosothiols (RSNOs).

In one aspect, the amount of the nitric oxide releasing compound is from about 0.1 weight percent to about 20 weight percent of the composition or about 0.1 weight percent, 0.5 weight percent, 1 weight percent, 2 weight percent, 4 weight percent, 6 weight percent, 8 weight percent, 10 weight percent, 12 weight percent, 14 weight percent, 16 weight percent, 18 weight percent, or 20 weight percent, where any value can be a lower and upper endpoint of a range (e.g., 4 weight percent to 14 weight percent).

In one aspect, the nitric oxide releasing compound includes a modified antibiotic compound including a nitric oxide release agent covalently attached to an antibiotic molecule. Having a single molecule with the combined functionalities of both of a stable NO donor and an antibiotic can be a very efficient approach for combating and preventing biofilm related infections.

The modified antibiotic compound can be a synthetic RSNO (e.g. S-nitroso-N-acetylpenicillamine (SNAP)) covalently attached to an antibiotic molecule (e.g. ampicillin) to create a novel dual functional antimicrobial agent, also referred to as a modified antibiotic compound. In other aspects, the nitric oxide releasing compound can be such as S-nitroso-glutathione, and S-nitroso-N-acetylcysteine, S-nitrosocysteine, S-nitrosopenicillamine, S-nitroso-B,D-glucose, S-nitrosocaptopril, S-nitrosocysteamine, and S-nitroso-3-mercapto-propanoic acid.

In one aspect, the antibiotic molecule is ampicillin. In other embodiments, the antibiotic molecule can be vancomycin, gentamicin, cephalexin, and the like.

In one aspect, the modified antibiotic compound when comprising SNAP and ampicillin is referred to herein as SNAPicillin. The terms “modified antibiotic compound” and “SNAPicillin” are intended to be used interchangeably, although “modified antibiotic compound” can also include other combinations of nitric oxide release agents and antibiotic molecules as can be appreciated by one of skill in the art. The structure of SNAPicillin is provided below.

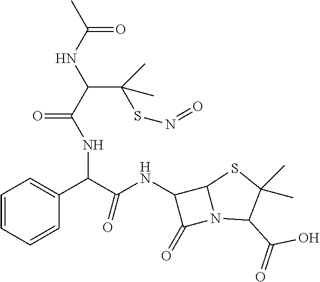 [SMILES: CC(=O)NC(C(=O)NC(C(=O)NC1C(=O)N2C1SC(C)(C)C2C(=O)O)c1ccccc1)C(C)(C)SN=O]

In one aspect, the modified antibiotic compound can be formed by covalently attaching a nitric oxide release agent to an antibiotic molecule. The attachment can be formed by mixing the nitric oxide release agent and the antibiotic molecule in a solvent then nitrosating the mixture. The nitrosation can occur through the excess addition of t-butyl nitrite or an acidified sodium nitrite solution to the mixture. The excess addition can be about 3 times molar excess of t-butyl nitrate with respect to the ampicillin quantity. Methods for producing the modified antibiotic compound useful as nitric oxide releasing compounds are describe din U.S. Pat. No. 11,220,516, which is incorporated by reference in its entirety.

### Bioactive Agents

The compositions described herein can include one or more bioactive agents depending upon the application of the composition. The amount of bioactive agent used in the compositions can also vary as well depending upon the application.

In one aspect, bioactive agent can include fluoride. The source of the fluoride can include fluoride salts. Examples of fluoride salts useful herein include sodium fluoride (NaF) or ammonium fluoride (NH₄F).

In another aspect, the bioactive agent includes metal nanoparticles (e.g., silver, zinc, copper, etc), antibiotics, antimicrobials, bactericides, antiseptics (e.g., chlorhexidine used in mouth washes), anti-inflammatory drugs, antimicrobial peptides, antimicrobial blue light (used in dental applications), enzymes, and ascorbic acid (vitamin C).

### Methods for Making the Compositions

Described herein are methods for making the compositions. In one aspect, the composition is produced by the process comprising
(a) mixing the polymer in a first solvent to produce a first composition;
(b) heating the first composition until a second homogeneous solution is produced;
(c) cooling the second homogeneous solution to produce a third homogeneous solution; and
(d) mixing the nitric oxide releasing compound with the third homogeneous solution to produce the composition.

In step (a), the polymer is mixed with a first solvent. The first solvent can be any solvent that solubilizes the polymer. In one aspect, the first solvent can be an alcohol such as, for example, methanol or ethanol. The mixture of polymer and first solvent can be heated in order to ensure the polymer is dissolved in the first solvent and the solution is homogeneous. In one aspect, the first composition in step (b) is heated from about 30° C. to about 80° C. After the homogeneous solution with the polymer has been cooled, the nitric oxide releasing compound is added to the homogeneous solution to produce the compositions described herein.

Prior to and/or after the addition of the nitric oxide releasing compound to the solution of polymer, additional components can be added. In one aspect, a compound that can reduce or prevent the decomposition of the nitric oxide releasing compound. In one aspect, the compound can be a biocompatible metal chelator such as, for example, an organic polyamine (i.e., an organic compound having two or more amine groups). Examples of metal chelators useful herein include, but are not limited to, ethylenediaminetetraacetic acid (EDTA) or bis(3-aminopropyl)amine (dipropylenetriamine (DPTA). In one aspect, the metal chelator is mixed with the alginate, the nitric oxide releasing compound, and the fluoride salt. In another aspect, a bioactive agent can be added prior to and/or after the addition of the nitric oxide releasing compound to the polymer solution.

The nitric oxide releasing compound is sensitive to light, which will ultimately cause the release of nitric oxide from the composition. In one aspect, the compositions are stored in the absence of light in order enhance shelf life and the efficiency of the compositions with respect to the amount of nitric oxide that can be released from the composition.

Exemplary methods for producing the compositions described herein are provided in the Examples.

### Methods of Use

The compositions described herein can be applied to any article or used to prepare an article where it is desirable reduce or prevent a bacterial infection in a subject. In one aspect, the article is a medical device that is used in oral or periodontal applications. The root of dental caries lies in the overactivity of bacteria on gums and teeth. Streptococcus mutans (S. mutans) and other dental pathogens colonize on the surface of teeth and form biofilms composed of protein, DNA, and polysaccharides. These biofilms, known as dental plaque, act as a protective barrier against antimicrobial treatments and allow the bacteria to proliferate uncontrolled. Although proper dental hygiene and regular brushing can keep these bacteria in balance and at bay, neglect of oral care can lead to excess plaque and in turn, overactive bacteria.

The compositions described herein address these issues, where the compositions are effective in delivering nitric oxide to the oral cavity of a subject. The compositions can kill bacteria in the oral cavity. Thus, the compositions are effective in treating or preventing bacterial infections, reducing or prevention the formation of biofilms in the oral cavity, and treating or preventing a periodontal disease in a subject.

In one aspect, the article is dental floss. Dental floss is commonly used to clean between teeth and gums, but its effectiveness in consistently reducing plaque and gum inflammation, even when used with toothbrushing, is often inconsistent due to its reliance on mechanical action. To address this issue, dental floss coated with the compositions described herein can actively target and eliminate bacteria responsible for plaque formation, going beyond its mechanical function. This can result in more effective plaque control and reduce the risk of dental issues like cavities and gum disease. In addition to dental floss, the compositions described herein can be applied to other periodontal articles such as dental sutures where it is desirable to release nitric oxide locally to prevent bacterial infection.

The compositions described herein can be formulated as a coating composition in a solvent so that when applied to a surface of an article and removal of the solvent, the polymer with the nitric oxide releasing compound remains on the surface of the article. For example, dental floss can be passed through a solution of the polymer and nitric oxide releasing compound followed by removal of solvent to produce a dental floss coated with a composition as described herein.

### Aspects

Aspect 1. An article comprising a composition comprising a polymer and a nitric oxide releasing compound, wherein the polymer comprises a polyacrylamide, polybetaine, a poloxamer, a polyester, a polypeptoid, a polyalkylene glycol, a polyalkylene, polylactic acid, polyglycolic acid, poly-d, I-lactic-co-glycolic acid (PLGA), glycerin, poly-N-vinylpyrrolidone, or any combination thereof.

Aspect 2. The article of Aspect 1, wherein the polymer comprises polyethylene glycol.

Aspect 3. The article of Aspect 2, wherein polyethylene glycol has an average molecular weight of from about 500 Da to about 10,000 Da.

Aspect 4. The article of Aspect 2 or 3, wherein polyethylene glycol comprises a first polyethylene glycol and a second polyethylene glycol, wherein the first polyethylene glycol and the second polyethylene glycol have a different average molecular weight.

Aspect 5. The article of Aspect 4, wherein the first polyethylene glycol has an average molecular weight of from about 500 Da to about 2,000 Da.

Aspect 6. The article of Aspect 4 or 5, wherein the second polyethylene glycol has an average molecular weight of from about 2,500 Da to about 4,000 Da.

Aspect 7. The article of any one of Aspects 4-6, wherein the weight ratio of the first polyethylene glycol to the second first polyethylene glycol is from 1:3 to 3:1.

Aspect 8. The article of any one of Aspects 2-7, wherein the polyethylene glycol has a viscosity of about 50 centipoise to about 500 centipoise.

Aspect 9. The article of any one of Aspects 1-8, wherein the nitric oxide releasing compound is a S-nitrosothiol conjugated polymer, a S-nitrosothiol modified-dendrimers; a S-nitrosothiol modified polysaccharide, a S-nitrosothiol modified nano/microparticle, a S-nitrosothiol modified-protein, a nitrate, a N-diazeniumdiolates (NONOate), or a S-nitrosothiol (RSNO).

Aspect 10. The article of any one of Aspects 1-8, wherein the nitric oxide releasing compound is a modified antibiotic compound comprising a nitric oxide release agent covalently attached to an antibiotic molecule

Aspect 11. The article of Aspect 10, wherein the nitric oxide release agent is S-nitroso-N-acetylpenicillamine, S-nitroso-glutathione, and S-nitroso-N-acetylcysteine, S-nitrosocysteine, S-nitrosopenicillamine, S-nitroso-B,D-glucose, S-nitrosocaptopril, S-nitrosocysteamine, and S-nitroso-3-mercapto-propanoic acid.

Aspect 12. The article of Aspect 10 or 11, wherein the antibiotic molecule is ampicillin, vancomycin, gentamicin, or cephalexin.

Aspect 13. The article of Aspect 10, wherein the modified antibiotic compound comprises S-nitroso-N-acetylpenicillamine covalently attached to ampicillin.

Aspect 14. The article of any one of Aspects 1-8, wherein the nitric oxide releasing compound is a S-nitrosothiol compound.

Aspect 15. The article of any one of Aspects 1-8, wherein the nitric oxide releasing compound is S-nitroso-N-acetyl-penicillamine, S-nitroso-N-acetylcysteine, S-nitroso-N-acetyl cysteamine, S-nitrosoglutathione, S-nitrosocysteamine-glutathione, methyl S-nitrosothioglycolate, nitrosated cysteine, S-nitroso-N-acetyl-l-cysteine ethyl ester (SNACET), S-nitroso-L-homocysteine, S-nitroso-L-cysteine, S-nitroso-albumin, S-nitrosocaptopril, or any combination thereof.

Aspect 16. The article of any one of Aspects 1-8, wherein the nitric oxide releasing compound is S-nitroso-N-acetyl-penicillamine.

Aspect 17. The article of any one of Aspects 1-16, wherein the nitric oxide releasing compound is from about 0.1 weight percent of the composition to about 20 weight percent of the composition.

Aspect 18. The article of any one of Aspects 1-17, wherein the composition further comprises a bioactive agent.

Aspect 19. The article of Aspect 18, wherein the bioactive agent comprises fluoride, metal nanoparticle, an antibiotic, an antimicrobial, bactericides, an antiseptic, an anti-inflammatory compound, an antimicrobial peptide, an enzyme, ascorbic acid, or any combination thereof.

Aspect 20. The article of any one of Aspects 1-19, wherein the composition is produced by the process comprising
(a) mixing the polymer in a first solvent to produce a first composition;
(b) heating the first composition until a second homogeneous solution is produced;
(c) cooling the second homogeneous solution to produce a third homogeneous solution; and
(d) mixing the nitric oxide releasing compound with the third homogeneous solution to produce the composition.

Aspect 21. The article of Aspect 20, wherein the first composition is heated from about 30° C. to about 80° C.

Aspect 22. The article of Aspect 20 or 21, wherein the first solvent comprises an alcohol.

Aspect 23. The article of Aspect 20 or 21, wherein the first solvent comprises methanol.

Aspect 24. The article of any one of Aspects 1-23, wherein at least one surface of the article is coated with the composition.

Aspect 25. The article of any one of Aspects 1-24, wherein the article comprises a medical device.

Aspect 26. The article of any one of Aspects 1-24, wherein the article comprises a dental or periodontal article or an on orthodontic device.

Aspect 27. The article of any one of Aspects 1-24, wherein the article comprises dental floss, a dental suture, a denture.

Aspect 28. A method for treating or preventing a bacterial infection in an oral cavity of a subject in need thereof comprising using the article in any one of Aspects 1-27.

Aspect 29. A method for treating or preventing a periodontal disease in a subject in need thereof comprising using to the article in any one of Aspects 1-27.

Aspect 30. A method for preventing or reducing the formation of biofilm or dental plaque in a subject in need thereof comprising using the article in any one of Aspects 1-27.

Aspect 31. The method of one of Aspects 28-30, wherein the article is dental floss.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Materials and Methods

### Materials

Ethylenediamine-tetraacetic acid (EDTA), methanol (MeOH), potassium phosphate dibasic, sodium nitrite, sulfuric acid (H₂SO₄), 3-(4,5-dimethylthiazol-2-yl)-2,5-diphenyltetrazolium bromide (MTT), and Luria Bertani (LB) broth and agar were purchased from Sigma-Aldrich (St. Louis, MO). Hydrochloric acid (HCl) and fetal bovine serum (FBS) were purchased from VWR (Radnor, PA). All buffers and other aqueous solutions were prepared using (18.2 MQ) Ultrapure water using an in-house distillation system from Mettler Toledo (Columbus, OH). Phosphate-buffered saline (0.1 mM PBS) containing 2.7 mM KCl, 138 mM NaCl, 1.8 mM KH₂PO₄, and 10 mM Na₂HPO₄ at pH 7.4 was used in all experiments unless otherwise stated. Brain heart infusion agar and broth were purchased from Mckesson Medical-Surgical (Irving, TX). S. mutans (ATCC 25175) and E. coli (ATCC 25922) were purchased from the American Type Culture Collection (ATCC, Manassas, VA). Human-derived osteoblast cell line hFOB 1.19 (ATCC CRL-11372), primary gingival fibroblasts (HGF) (ATCC PCS-201-018), fibroblast basal medium, and the associated fibroblast growth kit with low serum were also purchased from ATCC. Dulbecco's modified Eagle's medium with nutrient mixture F12 (1:1 by volume) was purchased from Thermo Fisher Scientific (Waltham, MA). Trypsin-EDTA, and sterile phosphate-buffered saline (0.1 mM) without calcium and magnesium was obtained from Corning (Corning, NY). REACH unflavored waxed nylon floss and Angzhili Dental removable teeth model with silica gel gums were purchased from Amazon.

### S-Nitroso-N-Acetylpenicillamine Synthesis

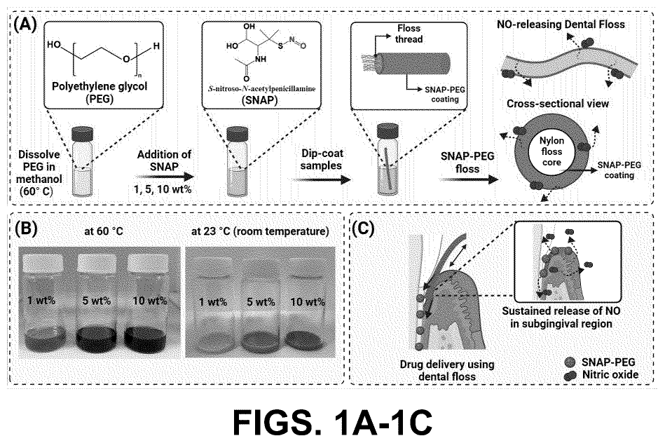

S-Nitroso-N-acetylpenicillamine (SNAP) was synthesized in a slightly modified procedure (FIG. 1A) based on prior work [47, 48]. The synthesis begins with the nitrosation of N-acetylpenicillamine (NAP) with an excess molar ratio of sodium nitrite in deionized water. NAP is combined with MeOH and concentrated HCl and H₂SO₄ which was stirred until the NAP is fully dissolved (1-2 min). Then NaNO₂ was added dropwise (<10 min) and finally the mixture was chilled while N₂ gas was blown over the solution to allow the SNAP crystals to precipitate out over 8 h. The green crystalline product is finally filtered and dried while protected from light exposure. The purity of SNAP was determined with a nitric oxide analyzer by injecting 30 μL 50 mM CuCl₂ and 1.5 μL 10 mM cysteine to PBS (without EDTA) for a total of 5000 μL then injecting 25 μL of SNAP and allowing the NO release to exhaust. This was repeated at least 3 times and the release profiles are then integrated to determine the total NO release compared to the theoretical yield. All SNAP used was ≥90% pure.

### Development of SNAP-PEG Coating

The NO-releasing coatings were constructed to incorporate the antimicrobial properties of NO into the dental floss for the prevention of periodontal infections. For this formulation, PEGs with average molecular weights of 1000 and 3350 and viscosities of 190 cP were combined, based on previous reports, in a 1:2 ratio in MeOH (2500 mg mL-1) [49]. The solution was then heated to 60° C. until the mixture was homogeneous and once cooled different weight percents of SNAP (1, 5, and 10 wt %) were added to the PEG/MeOH solution (FIG. 1B). A commercially available nylon floss was cut into approximately 8 cm strips and secured by one end. The samples were then dip coated by the different SNAP wt % solutions three times with intervals of one minute to allow for the coating to cool and adhere to the nylon substrate (FIG. 1C). The samples were allowed to dry for 24 h at room temperature undisturbed to allow the methanol to evaporate. For control samples, the PEG formulation was dissolved in methanol and used to coat the floss samples.

### Determination of Weight of SNAP Coated on Floss Using UV-Vis Spectroscopy

The individual coated samples were cut into 1 mm sections then suspended and agitated in 1 mL PBS buffer containing 100 μM EDTA to dissolve the SNAP-PEG coating. The concentration of SNAP was determined by using a UV-vis spectrophotometer (Cary 60, Agilent Technologies). The molar absorptivity of SNAP in PBS containing EDTA at 340 nm was determined to be 840 M⁻¹ cm⁻¹. The PEG mixture without SNAP was used as a blank control to confirm that the absorbance peak spectra at 340 nm is due to the presence of SNAP. The absorbance is then used to determine the weight of SNAP present in the samples from standard curve data of the SNAP used in synthesis.

### Coating Weight Uniformity

To quantify the uniformity of the coating weight distribution samples were cut into (1 cm) sections and weighed. Then the coating was fully dissolved in 1 mL of PBS with agitation and the floss was weighed again. The weight of the floss sample and coating combined was subtracted from the weight of the cleaned floss to find the weight of the coating. Data from the study is presented as the weight of SNAP in milligrams (mg) obtained from the mean of each sample group (control, 1, 5, and 10 wt % SNAP-PEG coated samples) normalized to the surface area of coated floss (N=3).

### Indirect Drug Delivery Efficiency

To determine drug delivery efficiency into the gum pocket, via a slightly modified method the tooth model was lightly lubricated with PBS 10 mM containing 100 PM EDTA, and then individually coated floss samples (4 mm length and 1 mm diameter) were passed back-and-forth three times in the periodontal pocket in either two of the front incisors of a tooth model. The tooth model was then swabbed in the gum pocket using a cotton-tipped stick that had been slightly wet with PBS containing 100 UM EDTA and placed back in a tube of PBS containing EDTA (1 mL) and agitated with a vortex device to dissolve and homogenize the floss coating. The SNAP concentration was then calculated by measuring the absorbance at 340 nm using a UV-vis spectrophotometer. The molar absorptivity at 340 nm was determined to be 1002 M⁻¹ cm⁻¹. The weight is reported as milligrams of SNAP deposited in the gum pocket.

### Surface Characterization

Images were collected using scanning electron microscopy (SEM) from Thermo Fisher, the Teneo FE-SEM with a current of 0.4 nA and the accelerating voltage was 5.0 kV. Samples were coated with 20 nm of gold-palladium with a Leica sputter coater before SEM imaging. The SEM images illustrate the effectiveness of the deposition of the coating. Images illustrate the coating before and after the sample has been deposited in the periodontal pocket of a tooth model. A medical tooth model with malleable silicone rubber gums and high-density polyethylene teeth was used to mimic the deposition of the coating between the teeth into the periodontal pocket in representational 3-dimensional space.

### NO Release Studies

### Total Nitrite Concentration to Estimate NO Concentration

To illustrate the decomposition of the SNAP-PEG coating in solution the NO concentrations were estimated via Griess assay by finding the total nitrite concentration present [50, 51]. To estimate NO concentrations via the Griess assay, 0.5 cm floss samples were submerged in 0.1 mM PBS containing 100 UM EDTA (1 mL) and incubated without agitation at 37° C. for 2, 4, 6, and 30 h. Aliquots (10 μL) of this sample were added to the Griess reagent (90 L, 22.22 mg mL-1) for a final concentration of 20 mg mL-1 to form a colorimetric product and the absorbance measured in each well was measured at 540 nm with a plate reader (BioTek Cytation 5 imaging reader). Sodium nitrite standards (0, 0.3125, 0.625, 1.25, 2.5, 5, 10, 20, 40 mM) were used to normalize the assay reactivity and associated absorbance. The molar absorptivity at 540 nm was determined to be 1240 M⁻¹ cm⁻¹.

### Nitric Oxide Analyzer

The experimentation was carried out to evaluate the NO release profile of each wt % SNAP-coated floss. Instantaneously the NO-release from the floss samples was measured by a Sievers 280i chemiluminescence Zysense Nitric Oxide Analyzer (NOA) 280i (Frederick, CO). The NOA was calibrated using a two-point calibration (0 and 45 ppm NO calibration gas). The NOA had a cell pressure of 6.4 Torr and a supply pressure of 10.9 Torr. The initial baseline NO release was in the range of 0-1 ppb. Samples were prepared by adding 200 μL of PBS buffer with 100 UM EDTA (10 mM 7.4 pH) solution to a KimTech wipe to keep the sample moist. These conditions mimic the physiological environment of the human mouth; samples were incubated at 37° C. and suspended so as to not directly touch the KimTech wipe. The NO released from the coated floss samples was immediately swept to the chemiluminescence detection chamber due to the flow of nitrogen gas (200 mL min-1). The samples are incubated at 37° C. between time points and the NO release is measured at 0, 2, 6, and 30 h to find the average ppb of NO for each sample. The NO flux is then determined by subtracting the baseline from the NOA from the release profile of the sample and converting the ppb to flux (x 10⁻¹⁰ mol min⁻¹ cm⁻²), normalizing the samples by surface area.

### Shelf-life Stability of SNAP-PEG Coating

To evaluate the stability of the SNAP-PEG coating, the coated floss samples were wrapped in a KimTech wipe and stored at room temperature (24° C.) in an airtight vial with desiccant protected from ambient light. The amount of remaining SNAP in the coating was measured using the same UV-vis spectroscopy method as described in section 2.4 to determine the absorbances of the samples compared to the initial levels of SNAP present. The samples were evaluated at four different time points (7, 14, 21, and 28 d) to determine the stability of the samples over a 28-d period in relevant medical storage conditions. The data is reported as the percent SNAP remaining on the coated floss after each timepoint normalized to the initial percent of SNAP in freshly prepared samples on day 0.

### Antimicrobial Activity of SNAP-PEG Coating

The ability of the coating to eliminate bacteria was characterized against S. mutans (Gram-positive cocci) and E. coli (Gram-negative rod), common bacteria associated with biofilm-related infections in the periodontal pocket. Bacteria most commonly found as early colonizers orally are streptococci which are anaerobic Gram-positive cocci, and anaerobic Gram-negative rods like E. coli [52]. The E. coli was grown in LB media and S. mutans in BHI media until the mid-log phase and then diluted in PBS. The bacteria suspension was washed by centrifuging the culture at 3500 RPM for 7 min and re-suspended into sterile PBS. Bacteria OD₆₀₀ was measured using a UV-vis spectrophotometer (Cary 60, Agilent Technologies). The bacteria culture was diluted to 0.1 OD and 100 μL of suspension was pipetted onto an agar plate and uniformly spread using a sterile cotton swab. Blank antibiotic disks were placed at equal distances from each other on the plate, and 200 μL pipetted with the coating solution for each weight percent of SNAP. As for controls, a paper disk with just 200 μL of PEG solution was added to the agar plate. The plates were incubated overnight at 37° C. and then the zone of inhibition was measured by finding the diameter of the zone of impeded bacterial growth surrounding the disk from three different directions and averaged. The results are presented as the average mm of zone±standard deviation (n≥4).

### Cytocompatibility Assessment

### Cell Culture Preparation

Cells were revived from cryopreserved stocks using complete media for each cell line following the manufacturers' recommendations. HGF cells were cultured in fibroblast basal medium supplemented with a growth kit containing fetal bovine serum (2%), ascorbic acid (50 μg/mL), recombinant human insulin (5 μg/mL), hydrocortisone hemisuccinate (1 μg/mL), recombinant human fibroblast growth factor b (5 ng/ml), L-glutamine (7.5 mM), and penicillin-streptomycin (10 U/mL). The hFOB cells were grown in media containing an equal volume of Ham's F12 medium to Dulbecco's modified Eagle's medium, further supplemented with L-glutamine (2.5 mM), fetal bovine serum (10%), and G418 antibiotic (0.3 mg/mL). Cells were incubated at 37° C. under a 5% CO₂-humidified atmosphere. Cells were treated with clean media every 48 h and grown to no greater than 80% sub-confluency. For the controlled release leachate testing, cells were detached via enzymatic treatment with trypsin (0.05% supplemented with 5 mM EDTA), centrifuged to collect the cell pellet (200 RCF, 5 min), and re-suspended to achieve a seeding density of 10,000 cells/well in culture-treated 96-well plates.

### Controlled Release Study for Cytocompatibility

Cytotoxicity screening of SNAP-PEG coated substrates was carried out following the International Organization for Standardization Protocol 10993-5:2009 with minor deviation [53]. In brief, coated floss segments (approximately 0.33 cm²) were dissolved in 1 mL of complete media for 24 h. Concurrently, cells are seeded onto 96-well plates (100 μL complete media per well) and incubated for 24 h. Afterward, leachate samples from each substrate classification are used to treat cells, replacing media with leachate-containing media (100 μL). Control wells were concurrently developed using clean complete media. Cells were grown for an additional 24 h. Following incubation, media was aspirated off from each well and replaced with MTT-containing media (0.5 mg/mL MTT, 100 μL). Cells were incubated for an additional 2 h. Subsequently, wells were aspirated of undissolved tetrazolium salt and the remaining formazan salt was dissolved in dimethyl sulfoxide (200 μL/well). Wells were read for absorbance at 570 nm with a reference reading at 690 nm. The percent cellular viability was then calculated relative to control wells as follows:

$$Relative Cell Viability ( \% ) = \frac{{OD}_{5 \; 70 , Treatment} - {OD}_{6 \; 90 , Treatment}}{{OD}_{5 \; 70 , Control} - {OD}_{690 , Control}} \times 100 \% \qquad ( 1 )$$

Final data are reported as the mean cellular viability±standard deviation (SD) (N=4 technical repeats across three independent passages).

### Statistical Analysis

The statistical analysis for all of the reported studies was done in GraphPad Prism 9 (GraphPad Software, San Diego, CA). For the NO flux determination a 2-way ANOVA with Tukey's multiple comparison test with an alpha value of 0.05 was used to determine any significant differences between the compositions. Additionally, for the remaining studies, comparisons of the different weight percentages of SNAP were determined by an ordinary one-way analysis of variance (ANOVA) with Tukey's method for multiple comparisons was used given values of p<0.05 to be significantly different.

### Results and Discussions

### Development of NO-Releasing Floss Coating

An antimicrobial coating containing nitric oxide (NO) donor SNAP and PEG polymer (SNAP-PEG) was devised to facilitate the release of antibacterial and anti-inflammatory NO into the periodontal pocket, presenting an inventive approach to prevent microbial infections (FIG. 1A). Polyethylene glycol (PEG) was chosen as the base material for this coating due to its excellent biocompatibility and its ability to dissolve in a variety of solvents. In this study, high-molecular-weight PEGs were employed, as they solidify at room temperature once the methanol solvent evaporates. This characteristic allows for the development of consistent coatings on a range of substrates (FIG. 1B). To illustrate the tunability of NO release rates, different weight percentages of SNAP (1, 5, and 10 wt %) were added to the PEG-methanol mixture. Subsequently, this SNAP-PEG blend was coated on a commercially available nylon dental floss using a simple dipcoating process. This integration of NO-releasing properties into the dental floss transforms it into a viable carrier for delivering NO donors to the subgingival region (FIG. 1C).

### Surface Characterization of NO-Releasing Dental Floss

### Determination of SNAP Payloads on Surface

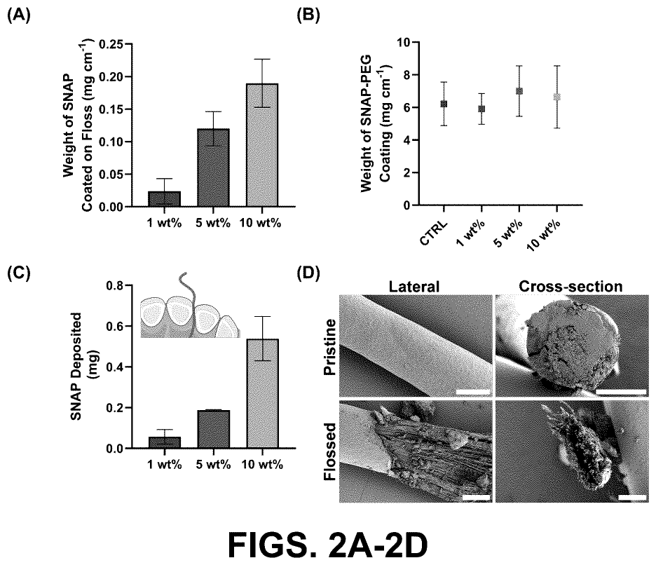

The NO donor SNAP was loaded in the PEG matrix by adding varying payloads (1, 5, and 10 wt %) during the fabrication process. The SNAP-PEG coating was then dip-coated onto the floss's surface and left to dry at room temperature (RT), facilitating the evaporation of methanol and the formation of a consistent coating. The amount of SNAP embedded into the coating was determined in order to evaluate the coating's ability to work as a targeted drug delivery system (FIG. 2A). The amount of SNAP in the coating was calculated via UV-Vis spectroscopy by dissolving 1 cm sections of floss in 1 mL PBS buffer containing 100 PM EDTA. Results from the study unveiled that the SNAP-PEG coating contained 0.089±0.015, 0.585±0.028, and 0.771±0.081 mg of SNAP per cm of floss for 1, 5, and 10 wt %, respectively. The coating had an increasing weight of SNAP embedded in the PEG matrix with an increasing weight percentage of SNAP added showing there was a range of SNAP concentrations stable in the PEG matrix. The coating demonstrated an increasing SNAP content within the PEG matrix as the weight percentage of SNAP added increased. Notably, while a small portion of SNAP underwent minimal degradation during the fabrication steps due to the presence of methanol and the drying process, bioactive levels of SNAP were still present on the floss. This study underscores the tunability of SNAP concentrations within the SNAP-PEG coating, an essential characteristic for achieving targeted drug delivery to the periodontal pocket.

The effectiveness of the SNAP-PEG coating as a precise drug delivery system relies on the uniformity of its application onto the nylon floss substrate. To assess this uniformity, measurements were taken of the floss's weight both before and after the deposition of the SNAP-PEG coating, and the weight of the coated floss was then subtracted from the weight of the nylon core (FIG. 2B-C). The average weight of the PEG coatings was found to be ca. 6.211±1.334, 5.899±0.945, 6.992±1.545, and 6.637±1.912 for the control (PEG without SNAP), 1, 5, and 10 wt % coatings, respectively. There were no significant differences observed among the average coating weights, and minimal variation was noted across the samples. These results highlight the efficiency and consistency of the coating process, particularly in the context of a rapid and straightforward fabrication method. Uniformity in the coated floss is of paramount importance for delivering consistent amounts of SNAP into the periodontal pocket. The study affirms the presence of such uniformity in SNAP-PEG coatings deposited onto the surface of the nylon floss, irrespective of the formulation used.

### Indirect Drug Delivery Efficiency of SNAP-PEG Coated Floss

To assess how easily the SNAP-PEG coating could be deposited into the subgingival region, an indirect drug delivery efficiency test was utilized. The drug delivery efficiency was estimated by flossing a malleable tooth model with SNAP-PEG coated floss and swabbing the tooth and periodontal pocket to collect the SNAP-PEG coating deposited. The deposited coating was then dissolved in 1 mL PBS buffer containing 100 μM EDTA and the weight of SNAP was calculated by reading the absorbance with UV-Vis spectroscopy. The results of the indirect drug delivery study indicated that, after flossing, 0.057±0.036, 0.187±0.003, and 0.539±0.109 mg of SNAP were deposited for 1, 5, and 10 wt % samples respectively, into the periodontal pocket of the tooth model after flossing. A direct correlation was evident between the amount of SNAP deposited in the subgingival region and the SNAP content within the SNAP-PEG coating. Notably, regardless of the weight percentage formulations, the SNAP-PEG-coated dental floss effectively delivered SNAP into the periodontal pocket of the model. These findings provide conclusive evidence of the SNAP-PEG coated floss's capability to deliver precise amounts of SNAP into the periodontal pocket.

This approach underscores the precision with which SNAP-PEG-coated dental floss can channel the NO donor specifically to the dental cavity. This offers significant advantages for targeted administration of antibacterial agents while minimizing systemic drug absorption. By directing NO precisely to the affected area through targeted delivery, the healing process can be significantly enhanced, providing a direct supply of NO to the infection site or damaged region. It is important to note that the quantity of SNAP within the PEG formulation can be customized based on the severity of the condition and the patient's requirements. Consequently, this methodology illustrates the potential for controlled and localized antimicrobial agent deposition offering versatile applications for both the treatment and prevention of periodontitis.

### Surface Analysis Using Scanning Electron Microscopy (SEM)

Scanning electron microscopy imaging was used to demonstrate how the coating is effortlessly inserted into the periodontal pocket through direct contact and minimal exertion (FIG. 2D). The images show the coating is relatively uniformly distributed on the floss, corroborating with the weight distribution data. After the coated floss samples were lightly mechanically flossed three times in either incisor of the tooth model, the fibers of the floss were then visible. The SEM imaging provided visual evidence that a substantial amount of SNAP was deposited into the periodontal pocket as seen in the flossed structure of the dental floss. It was also observed that the dental floss maintained its original physical attributes, ensuring that it could continue to function effectively as a tool for oral care while incorporating the beneficial properties of the SNAP-PEG coating. An excess amount of coating is applied to the floss to ensure thorough deposition within the oral pocket. Excessive microbial activity can lead to the development of pathological pockets around affected teeth, and this bioactive coating is designed to mitigate subgingival infections commonly associated with periodontal disease.

### NO Release Kinetics Under Physiological Conditions

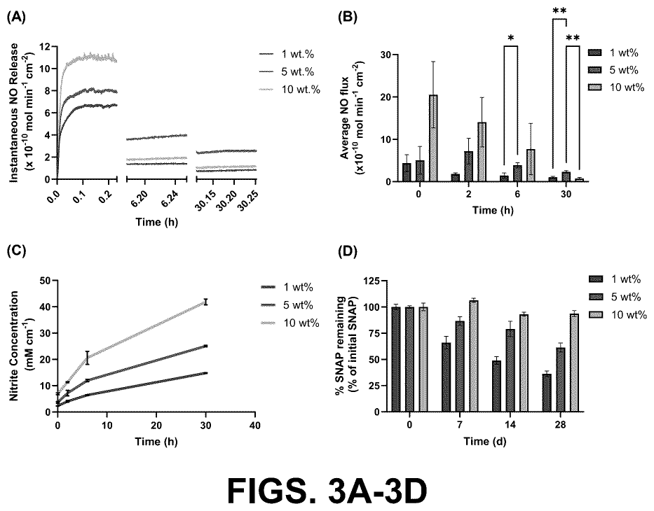

SNAP releases NO via thermal decomposition, metal ion catalysis, and photolysis when the light energy corresponds with the SNAP absorption bands at 340 nm and 590 nm [33]. To mimic the moist environment of the oral cavity, the NO release studies were conducted under humidified conditions, ensuring that the coating remained intact at each time point for a comprehensive assessment of the NO release characteristics without immediate dissolution. The extent of NO release was found to be influenced by the concentration of SNAP and the reactivity of the environment with embedded SNAP in the polymeric matrix. The average flux for each weight percentage (wt %) of SNAP measured depicted the NO release of the floss coating under simulated physiological conditions at various time points up to 30 h (FIG. 3A).

Similar to other materials utilizing SNAP as the NO donor, the initial evolution of NO release exhibited higher levels that gradually decreased over time. This observed rate of NO release can be attributed to the hydrophilic nature of PEG, known for its high water absorption capacity [57]. [60]. Throughout the study, the 10 wt % SNAP samples, on average, demonstrated the highest rates of NO release. The overall trends in the average NO flux were significantly influenced by the weight percentage of SNAP in the PEG coating. By the 30 h timepoint, the NO release from all samples diminished as the SNAP payload was depleted (Table 1). Previous research has established a correlation between higher NO flux and increased antibacterial efficiency [58]. [61]. Notably, the 10 wt % coatings displayed a higher cumulative average flux compared to the 1 and 5 wt % coatings (FIG. 3B). However, there was no significant difference in the average NO flux between the 5 and 10 wt % coatings at 6 and 30 h. The profiles representing instantaneous flux at 6 and 30 h for 5 wt % SNAP were greater than those for 10 wt %, which can be explained by the fact that instantaneous flux is representative of a single sample, and there was no significant difference in the average flux at those time points. Furthermore, the NO release demonstrates that the SNAP-PEG coating has the capability to release NO in humid conditions without significant SNAP loss before reaching bioactive levels of NO release, and this release can be sustained for up to 30 h.

TABLE 1
Average NO flux data over 30 h at different time points
for each weight percentage of SNAP in the floss coatings.
Average NO flux (×10⁻¹⁰ mol min⁻¹ cm⁻²)

| SNAP wt. % | Time (h) | | | |
|---|---|---|---|---|
| in PEG | 0 | 2 | 6 | 30 |
| 1 wt % | 3.97 ± 1.79 | 1.45 ± 0.19 | 1.19 ± 0.37 | 0.91 ± 0.20 |
| 5 wt % | 4.88 ± 2.70 | 6.89 ± 2.49 | 3.61 ± 0.43 | 2.21 ± 0.22 |
| 10 wt % | 23.4 ± 8.34 | 14.0 ± 4.78 | 7.68 ± 4.95 | 0.74 ± 0.19 |

Separate controlled release degradation studies in aqueous solutions with the coated floss samples were completed under static incubation at 37° C. to estimate the NO release with the total nitrite concentration from a Griess assay. The nitrite accumulation test is distinct from a SNAP accumulation study and estimates the NO release rather than the amount of SNAP leached into the solution. The samples were quickly dissolved into PBS with 100 μM EDTA solution, where it would continue to release NO in solution. It was observed that the total nitrite concentration had a similar release profile to the humid conditions detected for each timepoint up to 30 h (FIG. 3C). This data aligns with the NO release patterns observed through the chemiluminescence method, demonstrating the controlled degradation of the coating beyond just humid conditions. These methods described are used to illustrate the NO release profiles in various conditions the coating might be exposed to after it is deposited on teeth or subgingival tissue.

The SNAP-PEG coating demonstrated controlled release in both solutions in a controlled degradation study and in humid conditions which can be utilized for targeted drug delivery in the periodontal pocket. Due to SNAP's degradation under different conditions and NO's short half-life, effective control of the release conditions from the SNAP-PEG matrix is crucial to harnessing it as a targeted antimicrobial agent within the periodontal pocket. While dental floss is a widely accepted tool for oral hygiene, its potential as a vehicle for topical drug delivery has received limited attention in the literature. Previous research has shown multiple therapeutics can be integrated into the floss for targeted delivery of antibacterial drugs into the periodontal pocket including gold nanoparticles (AuNPs), povidone-iodine, and chlorhexidine, etc. [9, 62, 63]. These approaches have demonstrated effective antibacterial properties, showing promise in combating periodontal infections. However, one key concern with these methods is the stability of the drug coatings over time. Moreover, AuNPs can be expensive to produce, which could increase the overall cost of dental floss products. This may limit their accessibility to a broader population making it less user-friendly for individuals seeking routine oral care. In contrast, SNAP-PEG-coated dental floss is user-friendly and can easily be integrated into an individual's daily oral care routine. It is easy to synthesize, economical, and offers a unique advantage in terms of stability. The sustained and extended release of NO from SNAP-PEG coating highlights the long-lasting effectiveness of this material as a therapeutic agent for periodontal care. This characteristic is particularly valuable in the context of periodontal disease management, where continuous and reliable drug delivery is essential for preventing and treating bacterial infections in the oral cavity.

### Shelf-Life Stability of SNAP-PEG Coating

The success of biomaterials is highly dependent on their ability to retain their function for an extended time after fabrication. To evaluate the potential of the SNAP-PEG coating to be stored at room temperature (RT) for effective clinical translation, the percent SNAP remaining was determined with UV-vis spectroscopy after 28 d of storage (FIG. 3D). The floss coating was stored in a tightly closed vial, in the dark, and with desiccant to protect the coating from moisture. Findings from the storage stability analysis indicated that PEG coatings containing higher weight percentages of SNAP exhibited superior retention of the total loaded SNAP compared to coatings with lower weight percentages. For 5 and 10 wt % SNAP samples after the 7 d timepoint still had greater than 85% of remaining SNAP and 10 wt % had 93.65±2.82 percent remaining after 28 d. However, the 1 wt % samples degraded more quickly. The 1 wt % coating degraded to 65.94±6.00% remaining over 1 d and went as low as 36.23±2.82 on the 28 d timepoint. The 10 wt % coating is relatively stable at RT when shielded from light and humid conditions. Previous studies have shown that the incorporation of SNAP into a polymeric matrix enhances its stability as crystalline SNAP embedded in the polymer matrix is released more slowly than dissolved SNAP from the polymer matrix and it shields the RSNO donor from environmental conditions such as light and heat [38]. NO-releasing materials largely face the challenge of tuning the rate of NO release for their intended purpose as well as maintaining a stable shelf life in relevant medical conditions. However, by incorporating SNAP into the polymeric matrix, the risk of SNAP degradation or loss during storage or application is minimized, allowing for reliable and consistent delivery of the active compound. This increased stability contributes to the overall efficacy and reliability of the floss coating as a drug delivery system for patients.

### In Vitro Testing

### Antimicrobial Efficacy of SNAP-PEG Coating

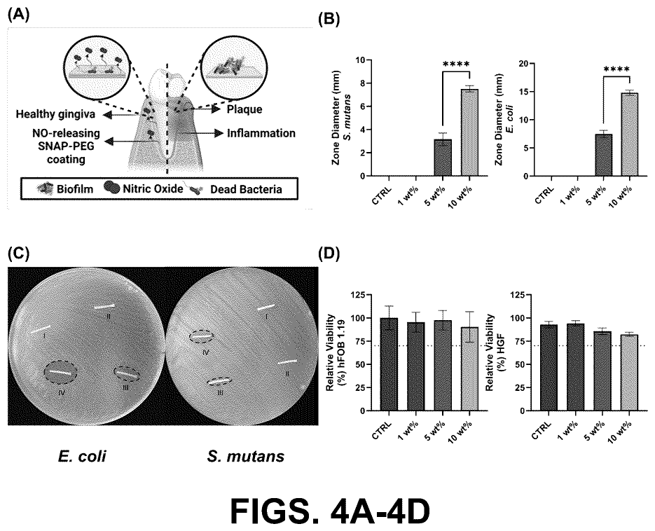

Periodontitis affects nearly half of the adult population in the United States, and, with a prevalence of about 11.2% worldwide, stands as the sixth most common human disease [55, 64]. This chronic inflammatory condition results in the deterioration of the tissues supporting the teeth and can ultimately lead to tooth loss, affecting speech, nutrition, and aesthetics [65]. Moreover, periodontitis has broader systemic implications, contributing to conditions like cardiovascular disease, and coronary heart disease, as it fosters systemic inflammation [66]. The main cause of periodontitis is the buildup of bacterial plaque biofilm in the subgingival area. This region is the gap between the tooth and the gingiva located beneath the gingival margin. The presence of bacterial biofilm disrupts the balance in the oral microbiome and triggers a destructive inflammatory immune response from the host. Consequently, plaque removal plays a crucial role in both preventing and treating periodontitis. Furthermore, given the susceptibility of pathogens to reestablish themselves in gum pockets, consistent daily plaque removal is essential for an effective treatment regimen. Conventional toothbrushes have limitations in reaching the spaces between teeth, thus requiring the use of specialized tools like dental floss for thorough cleaning of interdental regions. Although dental floss is a common tool in daily oral hygiene routines, it frequently falls short in reducing plaque and mitigating gum inflammation, even when combined with regular toothbrushing and oral rinsing. This limited efficacy is primarily attributed to the mechanical nature of dental floss [54, 55]. To overcome these limitations, a NO-releasing antimicrobial coating was integrated with dental floss for efficient delivery of NO into the subgingival region (FIG. 4A).

To assess its antimicrobial efficacy, the SNAP-PEG coating was subjected to zone of inhibition (ZOI) assays targeting both S. mutans (Gram-positive) and E. coli (Gram-negative). Notably, S. mutans, often present in the periodontal pocket, plays a pivotal role by generating acid that erodes enamel and creates a favorable environment for the colonization of other bacteria [67]. Similarly, E. coli is a facultative anaerobic Gram-negative bacteria present in the oral microbiome, with an ability to produce lipopolysaccharides (LPS) to increase the inflammatory action in the periodontal pocket [68-70]. These virulence factors such as LPS and peptidoglycan produced by the periodontopathic bacteria induce host responses, including the production of pro-inflammatory cytokines. The elimination of bacteria responsible for byproducts such as LPS including E. coli, will lower the levels of chemokines which are responsible for triggering chronic inflammation [71].

The results from the ZOI test of SNAP-PEG coating unveiled a significant difference in the diameter of inhibited zone of growth for each tested formulation containing 1, 5, and 10 wt % of SNAP, and no zone of inhibition was observed for the control PEG sample which was expected since PEG control coating lacked any inherent antibacterial mechanism of action. The increased zone of inhibition is directly correlated to the increasing concentration of SNAP available in the coating (FIG. 4B-C). The ZOI for S. mutans against the 10 wt % SNAP-PEG coating had approximately 2.2× and 1.3× greater ZOI than the 1 wt % and 5 wt %, respectively (p<0.05). Comparatively, the zone of inhibition for E. coli against the 10 wt % SNAP-PEG coating had approximately 1.9× and 1.2× greater ZOI than the 1 wt % and 5 wt %, respectively (p<0.05). The antibacterial assay results align with the NO release data from each formulation (FIG. 3B), showing a direct correlation between higher SNAP concentrations and increased levels of NO release. Increased NO release results in elevated levels of reactive oxygen species (ROS) within the bacterial environment, triggering processes such as membrane disruption, DNA lysis, and lipid peroxidation ultimately leading to bacterial death.

NO's gaseous nature facilitates easy penetration of bacterial membranes, damaging DNA and inactivating heme proteins involved in signal transduction [24]. Unlike specific antibiotics that target distinct pathways in different bacteria types, NO's multimechanistic and nonspecific action remains effective across all bacteria types [73]. Notably, NO exhibits potent antibacterial effects against a wide range of bacteria including both antibiotic-resistant and susceptible bacteria without inducing NO resistance [74, 75]. This surpasses the effectiveness of alternative antibacterial agents, such as gold nanoparticles and chlorhexidine, which are frequently integrated into dental floss for the management of periodontal infections [9, 62, 63]. Synthetic chemicals, such as chlorhexidine, may disrupt the natural oral microbiome equilibrium and have cytotoxic effects [76]. Conversely, NO is a naturally found molecule responsible for several regulatory functions in oral health.

NO disrupts biofilm formation and weakens established biofilms, making bacteria more susceptible to antibacterial agents and the host's immune system [74, 77]. Thus, NO-releasing materials offer numerous advantages over conventional chemical agents for managing periodontal disease. The controlled NO release from SNAP-PEG-coated dental floss showcases tunable drug loading and effective antibacterial properties, making it a user-friendly approach that could improve patient access to treatment.

### Cytocompatibility of NO-Releasing Floss

Ensuring the SNAP-PEG coating does not induce a cytotoxic response is as important as the level of antimicrobial activity for the goal of preventing periodontal infection propagation. A controlled degradation study for relative cell viability was conducted to determine the compatibility of the different SNAP-PEG coatings under extraction conditions. Two cell types were used, HGFs and hFOB 1.19's, with the results of contact testing summarized in FIG. 4D. HGFs are the most abundant structural cells in the periodontal pocket, being the foundational cells of connecting tissue and playing critical roles in inflammatory processes and wound healing [78]. Osteoblastic cells are responsible for both soft and hard tissue restoration needed for the treatment of periodontitis. Both cell types showed a relative cell viability greater than 70% showing a broad cytocompatibility of the material. Similar to other NO-releasing materials for dental applications capable of eradicating dental pathogens the SNAP-PEG floss is effective at concentrations not diminishing the viability of human gingival fibroblast cells [46, 79]. These results follow previous studies of NO-releasing materials, as it is known that low dosages of NO promote fibroblast proliferation and migration [80]. Minimizing acute adverse biological effects from the leachates from tissue-contacting materials is crucial to prevent further inflammation. This demonstrates the biocompatible properties of the SNAP-PEG floss and combined with the antimicrobial properties of the floss illustrates a promising material coating for preventing periodontitis.

### CONCLUSIONS

Periodontitis requires continuous maintenance by both patients and health professionals. Despite extensive research, existing approaches fall short of preventing the condition. To address these challenges, a simple and effective floss coating was fabricated. Introducing the advancement to patient care in order to improve patient compliance as well as the delivery and retention of SNAP into the subgingival region. The SNAP-PEG coated floss was able to effectively treat both Gram-negative and -positive bacteria commonly present in the periodontal tissue. The facile method to fabricate the coating includes the use of a NO donor, SNAP, which is activated by heat and light to eliminate bacteria that cause periodontitis. The NO donor SNAP is embedded into a PEG mixture at varying concentrations for a tunable and controlled NO release for 30 h. Among the different concentrations of SNAP incorporated into the PEG coating, the NO flux was determined using a chemiluminescence NO analyzer. The average flux was as high as 23.4±8.34 and maintained for 30 h illustrating the longevity of the material for flossing. The NO flux levels for the novel SNAP-PEG coatings were antimicrobial-relevant levels and shown to be a controlled release targeted for the periodontal pocket. Additionally, the antibacterial activity and cytocompatibility were measured in vitro. The SNAP-PEG coating exhibited broad-spectrum antimicrobial action against both S. mutans (Gram-positive) and E. coli (Gram-negative) in the zone of inhibition studies. All formulations of the SNAP-PEG coating tested were deemed cytocompatible against HGF and hFoB cells, as shown with a controlled release compatibility study. The coating presented increases the accessibility to dental care for patients as well as minimizes the need for mechanical force required to floss for those with lower dexterity with a bioactive additive. The increased accessibility to dental care the coating presents is expected to significantly minimize the heavy financial burden of periodontitis. The coating technology can also be applied for different applications, including dental sutures [81, 82], as the coating is independent of the substrate. The ease of synthesis and fabrication, capacity of the tunable NO release and coating deposition, antimicrobial activity, cytocompatibility, and shelf-life stability make the SNAP-PEG floss coating a promising new solution for the prevention of periodontitis without the need for surgical intervention.

It should be emphasized that the above-described embodiments of the present disclosure are merely possible examples of implementations and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

### REFERENCES ADDIN EN.REFLIST

[1] World Health Organization, Global oral health status report: towards universal health coverage for oral health by 2030, in: W. H. Organization (Ed.) 2022, p. 100.
[2] T. Kwon, I. B. Lamster, L. Levin, Current concepts in the management of periodontitis, International Dental Journal 71 (6) (2021) 462-476.
[3] S. A. Hienz, S. Paliwal, S. Ivanovski, Mechanisms of Bone Resorption in Periodontitis, Journal of Immunology Research 2015 (2015).
[4] N. J. Kassebaum, E. Bernabe, M. Dahiya, B. Bhandari, C. J. Murray, W. Marcenes, Global burden of severe periodontitis in 1990-2010: a systematic review and meta-regression, J Dent Res 93 (11) (2014) 1045-53.
[5] J. Botelho, V. Machado, Y. Leira, L. Proença, L. Chambrone, J. J. Mendes, Economic burden of periodontitis in the United States and Europe: An updated estimation, Journal of Periodontology 93 (3) (2022) 373-379.
[6] P. P. Hujoel, J. Cunha-Cruz, D. W. Banting, W. J. Loesche, Dental Flossing and Interproximal Caries: a Systematic Review, Journal of Dental Research 85 (4) (2006) 298-305.
[7] M. Salas, A. Mcclellan, Macneill, Sr., K. Satheesh, C. Cobb, Interproximal cervical lesions caused by incorrect flossing technique, International Journal of Dental Hygiene 10 (2) (2012) 83-85.
[8] M. M. Meghil, F. Rueggeberg, A. El-Awady, B. Miles, F. Tay, D. Pashley, C. W. Cutler, Novel Coating of Surgical Suture Confers Antimicrobial Activity Against Porphyromonas gingivalis and Enterococcus faecalis, Journal of Periodontology 86 (6) (2015) 788-794.
[9] K. Kaewiad, T. Nakpheng, T. Srichana, Dental floss impregnated with povidone-iodine coated with Eudragit L-100 as an antimicrobial delivery system against periodontal-associated pathogens, Journal of Medical Microbiology 69 (2) (2020) 298-308.
[10] S. Boes, H. S. Gill, Drug-Coated Floss to Treat Gum Diseases: In Vitro and In Vivo Characterization, Acs Applied Materials & Interfaces 14 (25) (2022) 28663-28670.
[11] S. Boese, H. S. Gill, Coated floss for drug delivery into the gum pocket, Int J Pharmaceut 606 (2021) 120855.
[12] E. Varoni, M. Tarce, G. Lodi, A. Carrassi, Chlorhexidine (CHX) in dentistry: state of the art, Minerva Stomatol 61 (9) (2012) 399-419.
[13] A. Herczegh, B. Csák, E. Dinya, A. Moldován, Á. Ghidán, B. Palcsó, Z. M. Lohinai, Short- and long term antibacterial effects of a single rinse with different mouthwashes: A randomized clinical trial, Heliyon 9 (4) (2023).
[14] L. Peng, L. Chang, X. Liu, J. Lin, H. Liu, B. Han, S. Wang, Antibacterial property of a polyethylene glycol-grafted dental material, ACS applied materials & interfaces 9 (21) (2017) 17688-17692.
[15] H. Ahmadi, A. Ebrahimi, F. Ahmadi, Antibiotic therapy in dentistry, International journal of dentistry 2021 (2021).
[16] S. Saidin, M. A. Jumat, N. A. A. M. Amin, A. S. S. Al-Hammadi, Organic and inorganic antibacterial approaches in combating bacterial infection for biomedical application, Materials Science and Engineering: C 118 (2021) 111382.
[17] R. Mohanty, S. J. Asopa, M. D. Joseph, B. Singh, J. P. Rajguru, K. Saidath, U. Sharma, Red complex: polymicrobial conglomerate in oral flora: a review, Journal of family medicine and primary care 8 (11) (2019) 3480.
[18] S. M. Deupree, M. H. Schoenfisch, Morphological analysis of the antimicrobial action of nitric oxide on Gram-negative pathogens using atomic force microscopy, Acta biomaterialia 5 (5) (2009) 1405-1415.
[19] Q. Gao, X. Zhang, W. Yin, D. Ma, C. Xie, L. Zheng, X. Dong, L. Mei, J. Yu, C. Wang, Functionalized MoS2 nanovehicle with near-infrared laser-mediated nitric oxide release and photothermal activities for advanced bacteria-infected wound therapy, Small 14 (45) (2018) 1802290.
[20] F. Rong, Y. Tang, T. Wang, T. Feng, J. Song, P. Li, W. Huang, Nitric oxide-releasing polymeric materials for antimicrobial applications: a review, Antioxidants 8 (11) (2019) 556.
[21] H. Massoumi, R. Kumar, M. K. Chug, Y. Qian, E. J. Brisbois, Nitric Oxide Release and Antibacterial Efficacy Analyses of S-Nitroso-N-Acetyl-Penicillamine Conjugated to Titanium Dioxide Nanoparticles, ACS applied bio materials 5 (5) (2022) 2285-2295.
[22] C. F. Nathan, J. B. Hibbs Jr, Role of nitric oxide synthesis in macrophage antimicrobial activity, Current opinion in immunology 3 (1) (1991) 65-70.
[23] M. L. Jones, J. G. Ganopolsky, A. Labbé, C. Wahl, S. Prakash, Antimicrobial properties of nitric oxide and its application in antimicrobial formulations and medical devices, Applied microbiology and biotechnology 88 (2010) 401-407.
[24] F. C. Fang, Perspectives series: host/pathogen interactions. Mechanisms of nitric oxide-related antimicrobial activity, J Clin Invest 99 (12) (1997) 2818-25.
[25] B. J. Privett, A. D. Broadnax, S. J. Bauman, D. A. Riccio, M. H. Schoenfisch, Examination of bacterial resistance to exogenous nitric oxide, Nitric oxide 26 (3) (2012) 169-173.
[26] D. A. Wink, K. S. Kasprzak, C. M. Maragos, R. K. Elespuru, M. Misra, T. M. Dunams, T. A. Cebula, W. H. Koch, A. Andrews, J. S. Allen, DNA deaminating ability and genotoxicity of nitric oxide and its progenitors, Science 254 (5034) (1991) 1001-1003.
[27] L. Yang, E. S. Feura, M. J. R. Ahonen, M. H. Schoenfisch, Nitric oxide-releasing macromolecular scaffolds for antibacterial applications, Advanced healthcare materials 7 (13) (2018) 1800155.
[28] T. Yang, A. N. Zelikin, R. Chandrawati, Progress and promise of nitric oxide-releasing platforms, Advanced Science 5 (6) (2018) 1701043.
[29] P. G. Wang, M. Xian, X. Tang, X. Wu, Z. Wen, T. Cai, A. J. Janczuk, Nitric oxide donors: chemical activities and biological applications, Chemical reviews 102 (4) (2002) 1091-1134.
[30] E. J. Brisbois, H. Handa, T. C. Major, R. H. Bartlett, M. E. Meyerhoff, Long-term nitric oxide release and elevated temperature stability with S-nitroso-N-acetylpenicillamine (SNAP)-doped Elast-eon E2As polymer, Biomaterials 34 (28) (2013) 6957-6966.
[31] S. P. Hopkins, J. Pant, M. J. Goudie, C. Schmiedt, H. Handa, Achieving Long-Term Biocompatible Silicone via Covalently Immobilized <i>S</i>-Nitroso-<i>N</i>-acetylpenicillamine (SNAP) That Exhibits 4 Months of Sustained Nitric Oxide Release, ACS Applied Materials & Interfaces 10 (32) (2018) 27316-27325.
[32] M. K. Chug, C. Feit, E. J. Brisbois, Increasing the Lifetime of Insulin Cannula with Antifouling and Nitric Oxide Releasing Properties, ACS Applied Bio Materials (2019).
[33] P. N. Coneski, M. H. Schoenfisch, Nitric oxide release: part III. Measurement and reporting, Chem. Soc. Rev. 41 (10) (2012) 3753-3758.
[34] M. K. Chug, E. Brisbois, Smartphone compatible nitric oxide releasing insert to prevent catheter-associated infections, Journal of Controlled Release: Official Journal of the Controlled Release Society (2022) S0168-3659 (22) 00385.
[35] R. P. Kark, D. C. Poskanzer, J. D. Bullock, G. Boylen, Mercury poisoning and its treatment with N-acetyl-D, L-penicillamine, New England Journal of Medicine 285 (1) (1971) 10-16.
[36] V. Parameshvara, Mercury poisoning and its treatment with N-acetyl-D, L-penicillamine, Occupational and Environmental Medicine 24 (1) (1967) 73-76.
[37] M. Jones, A. Weaver, W. Weller, The relative effectiveness of some chelating agents as antidotes in acute cadmium poisoning, Research Communications in Chemical Pathology and Pharmacology 22 (3) (1978) 581-588.
[38] Y. Wo, Z. Li, E. J. Brisbois, A. Colletta, J. Wu, T. C. Major, C. Xi, R. H. Bartlett, A. J. Matzger, M. E. Meyerhoff, Origin of long-term storage stability and nitric oxide release behavior of CarboSil polymer doped with S-nitroso-N-acetyl-d-penicillamine, ACS applied materials & interfaces 7 (40) (2015) 22218-22227.
[39] R. Kumar, M. K. Chug, E. J. Brisbois, Long-Term Storage Stability and Nitric Oxide Release Behavior of (N-Acetyl-S-nitrosopenicillaminyl)-S-nitrosopenicillamine-Incorporated Silicone Rubber Coatings, ACS Appl. Mater. Interfaces (2022).
[40] C. A. Joseph, C. W. McCarthy, A. G. Tyo, K. R. Hubbard, H. C. Fisher, J. A. Altscheffel, W. He, R. Pinnaratip, Y. Liu, B. P. Lee, Development of an injectable nitric oxide releasing poly(ethylene)glycol-Fibrin adhesive hydrogel, ACS biomaterials science & engineering 5 (2) (2018) 959-969.
[41] M. Ganzarolli de Oliveira, S-Nitrosothiols as platforms for topical nitric oxide delivery, Basic & clinical pharmacology & toxicology 119 (2016) 49-56.
[43] A. B. Seabra, G. F. P. de Souza, L. L. da Rocha, M. N. Eberlin, M. G. de Oliveira, S-nitrosoglutathione incorporated in poly(ethylene glycol) matrix: potential use for topical nitric oxide delivery, Nitric Oxide 11 (3) (2004) 263-272.
[43] S. M. Shishido, M. G. de Oliveira, Polyethylene glycol matrix reduces the rates of photochemical and thermal release of nitric oxide from S-nitroso-N-acetylcysteine, Photochemistry and photobiology 71 (3) (2000) 273-280.
[44] L. Yang, F. Teles, W. Gong, S. A. Dua, L. Martin, M. H. Schoenfisch, Antibacterial action of nitric oxide-releasing hyperbranched polymers against ex vivo dental biofilms, Dental Materials 36 (5) (2020) 635-644.
[45] A. W. Carpenter, K. P. Reighard, J. E. Saavedra, M. H. Schoenfisch, O 2-Protected diazeniumdiolate-modified silica nanoparticles for extended nitric oxide release from dental composites, Biomaterials science 1 (5) (2013) 456-459.
[46] C. J. Backlund, B. V. Worley, M. H. Schoenfisch, Anti-biofilm action of nitric oxide-releasing alkyl-modified poly(amidoamine) dendrimers against Streptococcus mutans, Acta biomaterialia 29 (2016) 198-205.
[47] L. Field, R. V. Dilts, R. Ravichandran, P. G. Lenhert, G. E. Carnahan, An unusually stable thionitrite from N-acetyl-D,L-penicillamine; X-ray crystal and molecular structure of 2-(acetylamino)-2-carboxy-1,1-dimethylethyl thionitrite, Journal of the Chemical Society, Chemical Communications (6) (1978) 249.
[48] E. J. Brisbois, T. C. Major, M. J. Goudie, R. H. Bartlett, M. E. Meyerhoff, H. Handa, Improved hemocompatibility of silicone rubber extracorporeal tubing via solvent swelling-impregnation of S-nitroso-N-acetylpenicillamine (SNAP) and evaluation in rabbit thrombogenicity model, Acta Biomaterialia 37 (2016) 111-119.
[49] M. A. Bowen, Medicated dental floss and method of preparation, Google Patents, 1997.
[50] J. Sun, X. Zhang, M. Broderick, H. Fein, Measurement of nitric oxide production in biological systems by using Griess reaction assay, Sensors 3 (8) (2003) 276-284.
[51] K. S. Bohl, J. L. West, Nitric oxide-generating polymers reduce platelet adhesion and smooth muscle cell proliferation, Biomaterials 21 (22) (2000) 2273-2278.
[52] H. Surapaneni, P. S. Yalamanchili, M. H. Basha, S. Potluri, N. Elisetti, M. K. Kumar, Antibiotics in dental implants: A review of literature, Journal of pharmacy & bioallied sciences 8 (Suppl 1) (2016) S28.
[53] I. S. O. Standards, ISO 10993-5:2009 (E). Biological evaluation of medical devices-Part 5: Tests for cytotoxicity in vitro, International Organizational for Standardization Geneve, 2009.
[54] M. S. Cepeda, R. Weinstein, C. Blacketer, M. C. Lynch, Association of flossing/interdental cleaning and periodontitis in adults, Journal of clinical periodontology 44 (9) (2017) 866-871.
[55] N. Kassebaum, E. Bernabé, M. Dahiya, B. Bhandari, C. Murray, W. Marcenes, Global burden of severe periodontitis in 1990-2010: a systematic review and meta-regression, Journal of dental research 93 (11) (2014) 1045-1053.
[56] E. J. Brisbois, M. Kim, X. Wang, A. Mohammed, T. C. Major, J. Wu, J. Brownstein, C. Xi, H. Handa, R. H. Bartlett, Improved hemocompatibility of multilumen catheters via nitric oxide (NO) release from S-nitroso-N-acetylpenicillamine (SNAP) composite filled lumen, ACS applied materials & interfaces 8 (43) (2016) 29270-29279.
[57] R. Vercelino, T. M. Cunha, E. S. Ferreira, F. Q. Cunha, S. H. Ferreira, M. G. De Oliveira, Skin vasodilation and analgesic effect of a topical nitric oxide-releasing hydrogel, Journal of Materials Science: Materials in Medicine 24 (9) (2013) 2157-2169.
[58] S. Ali, A. S. Solano, A. L. Gonzales, P. Thakore, V. Krishnan, E. Yamasaki, S. Earley, Nitric oxide signals through IRAG to inhibit TRPM4 channels and dilate cerebral arteries, Function 2 (6) (2021) zqab051.
[59] Q. Zhang, M. E. Meyerhoff, Nitric oxide release for enhanced biocompatibility and analytical performance of implantable electrochemical sensors, Electroanalysis 33 (9) (2021) 1997-2015.
[60] E. J. Brisbois, J. Bayliss, J. F. Wu, T. C. Major, C. W. Xi, S. C. Wang, R. H. Bartlett, H. Handa, M. E. Meyerhoff, Optimized polymeric film-based nitric oxide delivery inhibits bacterial growth in a mouse burn wound model, ACTA BIOMATERIALIA 10 (10) (2014) 4136-4142.
[61] S. P. Nichols, M. H. Schoenfisch, Nitric oxide flux-dependent bacterial adhesion and viability at fibrinogen-coated surfaces, Biomaterials Science 1 (11) (2013) 1151.
[62] M. Nakajima, N. Nakajima, J. Guo, S. Mitragotri, Engineering of bioactive nanocomplexes on dental floss for targeted gingival therapy, Bioengineering & Translational Medicine 8 (2) (2023) e10452.
[63] N. Traitanon, S. Dechkunakorn, P. Tantivitayakul, P. Punnakitikashem, W. Wichai, P. P. Whitis, N. Anuwongnukroh, Antibacterial Effect of Gold Nanoparticles Coated Dental Floss against Cariogenic Bacteria, Key Engineering Materials, Trans Tech Publ, 2021, pp. 293-300.
[64] P. I. Eke, B. A. Dye, L. Wei, G. D. Slade, G. O. Thornton-Evans, W. S. Borgnakke, G. W. Taylor, R. C. Page, J. D. Beck, R. J. Genco, Update on prevalence of periodontitis in adults in the United States: NHANES 2009 to 2012, Journal of periodontology 86 (5) (2015) 611-622.
[65] M. C. Ferreira, A. Dias-Pereira, L. Branco-de-Almeida, C. Martins, S. Paiva, Impact of periodontal disease on quality of life: a systematic review, Journal of periodontal research 52 (4) (2017) 651-665.
[66] M. Sanz, A. Marco del Castillo, S. Jepsen, J. R. Gonzalez-Juanatey, F. D′Aiuto, P. Bouchard, I. Chapple, T. Dietrich, I. Gotsman, F. Graziani, Periodontitis and cardiovascular diseases: Consensus report, Journal of clinical periodontology 47 (3) (2020) 268-288.
[67] N. Takahashi, Microbial ecosystem in the oral cavity: Metabolic diversity in an ecological niche and its relationship with oral diseases, International Congress Series 1284 (2005) 103-112.
[68] H. Li, C. Gao, L. Tang, C. Wang, Q. Chen, Q. Zheng, S. Yang, S. Sheng, X. Zan, Lysozyme (Lys), Tannic Acid (TA), and Graphene Oxide (GO) Thin Coating for Antibacterial and Enhanced Osteogenesis, ACS Applied Bio Materials 3 (1) (2020) 673-684.
[69] X. Wang, H. Fan, F. Zhang, S. Zhao, Y. Liu, Y. Xu, R. Wu, D. Li, Y. Yang, L. Liao, H. Zhu, X. Wang, Antibacterial Properties of Bilayer Biomimetic Nano-ZnO for Dental Implants, ACS Biomaterials Science &amp; Engineering 6 (4) (2020) 1880-1886.
[70] L. M. E. Bright, M. R. S. Garren, M. Ashcraft, A. Kumar, H. Husain, E. J. Brisbois, H. Handa, Dual Action Nitric Oxide and Fluoride lon-Releasing Hydrogels for Combating Dental Caries, Acs Applied Materials & Interfaces 14 (19) (2022) 21916-21930.
[71] N. Takahashi, J. Washio, G. Mayanagi, Metabolomic approach to oral biofilm characterization-A future direction of biofilm research, Journal of Oral Biosciences 54 (3) (2012) 138-143.
[72] M. K. Chug, H. Massoumi, Y. Wu, E. J. Brisbois, Prevention of medical device infections via multi-action nitric oxide and chlorhexidine diacetate releasing medical grade silicone biointerfaces, J. Biomed. Mater. Res. A 110 (6) (2022) 1263-1277.
[73] M. K. Chug, E. J. Brisbois, Recent Developments in Multifunctional Antimicrobial Surfaces and Applications toward Advanced Nitric Oxide-Based Biomaterials, ACS Materials Au 2 (5) (2022) 525-551.
[74] K. R. Rouillard, O. P. Novak, A. M. Pistiolis, L. Yang, M. J. Ahonen, R. A. McDonald, M. H. Schoenfisch, Exogenous nitric oxide improves antibiotic susceptibility in resistant bacteria, ACS Infectious Diseases 7 (1) (2020) 23-33.
[75] M. K. Chug, L. Griffin, M. R. Garren, E. Tharp, G. Nguyen, H. Handa, E. J. Brisbois, Antimicrobial Efficacy of a Nitric Oxide-Releasing Ampicillin Conjugate Catheter Lock Solution on Clinically-Isolated Antibiotic-Resistant Bacteria, Biomater. Sci. (2023).
[76] H.-D. Müller, S. Eick, A. Moritz, A. Lussi, R. Gruber, Cytotoxicity and antimicrobial activity of oral rinses in vitro, BioMed Res. Int. 2017 (2017).
[77] N. Barraud, M. J Kelso, S. A Rice, S. Kjelleberg, Nitric oxide: a key mediator of biofilm dispersal with applications in infectious diseases, Current pharmaceutical design 21 (1) (2015) 31-42.
[78] T. Ara, K. Kurata, K. Hirai, T. Uchihashi, T. Uematsu, Y. Imamura, K. Furusawa, S. Kurihara, P. L. Wang, Human gingival fibroblasts are critical in sustaining inflammation in periodontal disease, Journal of periodontal research 44 (1) (2009) 21-27.
[79] L. Yang, M. H. Schoenfisch, Nitric Oxide-Releasing Hyperbranched Polyaminoglycosides for Antibacterial Therapy, ACS Applied Bio Materials 1 (4) (2018) 1066-1073.
[80] G. Han, L. N. Nguyen, C. Macherla, Y. Chi, J. M. Friedman, J. D. Nosanchuk, L. R. Martinez, Nitric oxide-releasing nanoparticles accelerate wound healing by promoting fibroblast migration and collagen deposition, The American journal of pathology 180 (4) (2012) 1465-1473.
[81] X. X. Deng, M. Qasim, A. Ali, Engineering and polymeric composition of drug-eluting suture: A review, Journal of Biomedical Materials Research Part A 109 (10) (2021) 2065-2081.
[82] Z. W. Ma, Z. Yang, Q. M. Gao, G. Y. Bao, A. Valiei, F. Yang, R. Huo, C. Wang, G. L. Song, D. L. Ma, Z. H. Gao, J. Y. Li, Bioinspired tough gel sheath for robust and versatile surface functionalization, Science Advances 7 (15) (2021).

1. An article comprising a composition comprising a polymer and a nitric oxide releasing compound, wherein the polymer comprises a polyacrylamide, polybetaine, a poloxamer, a polyester, a polypeptoid, a polyalkylene glycol, a polyalkylene, polylactic acid, polyglycolic acid, poly-d, I-lactic-co-glycolic acid (PLGA), glycerin, poly-N-vinylpyrrolidone, or any combination thereof.

2. The article of claim 1, wherein the polymer comprises polyethylene glycol.

3. The article of claim 2, wherein polyethylene glycol comprises a first polyethylene glycol and a second polyethylene glycol, wherein the first polyethylene glycol and the second polyethylene glycol have a different average molecular weight.

4. The article of claim 3, wherein the first polyethylene glycol has an average molecular weight of from about 500 Da to about 2,000 Da and the second polyethylene glycol has an average molecular weight of from about 2,500 Da to about 4,000 Da.

5. The article of claim 3, wherein the weight ratio of the first polyethylene glycol to the second first polyethylene glycol is from 1:3 to 3:1.

6. The article of claim 1, wherein the nitric oxide releasing compound is a S-nitrosothiol conjugated polymer, a S-nitrosothiol modified-dendrimers; a S-nitrosothiol modified polysaccharide, a S-nitrosothiol modified nano/microparticle, a S-nitrosothiol modified-protein, a nitrate, a N-diazeniumdiolates (NONOate), or a S-nitrosothiol (RSNO).

7. The article of claim 1, wherein the nitric oxide releasing compound is a modified antibiotic compound comprising a nitric oxide release agent covalently attached to an antibiotic molecule

8. The article of claim 7, wherein the nitric oxide release agent is S-nitroso-N-acetylpenicillamine, S-nitroso-glutathione, and S-nitroso-N-acetylcysteine, S-nitrosocysteine, S-nitrosopenicillamine, S-nitroso-B,D-glucose, S-nitrosocaptopril, S-nitrosocysteamine, and S-nitroso-3-mercapto-propanoic acid and the antibiotic molecule is ampicillin, vancomycin, gentamicin, or cephalexin.

9. The article of claim 7, wherein the modified antibiotic compound comprises S-nitroso-N-acetylpenicillamine covalently attached to ampicillin.

10. The article of claim 1, wherein the nitric oxide releasing compound is a S-nitrosothiol compound.

11. The article of claim 1, wherein the nitric oxide releasing compound is S-nitroso-N-acetyl-penicillamine, S-nitroso-N-acetylcysteine, S-nitroso-N-acetyl cysteamine, S-nitrosoglutathione, S-nitrosocysteamine-glutathione, methyl S-nitrosothioglycolate, nitrosated cysteine, S-nitroso-N-acetyl-I-cysteine ethyl ester (SNACET), S-nitroso-L-homocysteine, S-nitroso-L-cysteine, S-nitroso-albumin, S-nitrosocaptopril, or any combination thereof.

12. The article of claim 1, wherein the nitric oxide releasing compound is S-nitroso-N-acetyl-penicillamine.

13. The article of claim 1, wherein the nitric oxide releasing compound is from about 0.1 weight percent of the composition to about 20 weight percent of the composition.

14. The article of claim 1, wherein the composition further comprises a bioactive agent selected from the group consisting of fluoride, metal nanoparticle, an antibiotic, an antimicrobial, bactericides, an antiseptic, an anti-inflammatory compound, an antimicrobial peptide, an enzyme, ascorbic acid, and any combination thereof.

15. The article of claim 1, wherein the composition is produced by the process comprising
(a) mixing the polymer in a first solvent to produce a first composition;
(b) heating the first composition until a second homogeneous solution is produced;
(c) cooling the second homogeneous solution to produce a third homogeneous solution; and
(d) mixing the nitric oxide releasing compound with the third homogeneous solution to produce the composition.

16. The article of claim 1, wherein the article comprises a medical device.

17. The article of claim 1, wherein the article comprises a dental or periodontal article, an orthodontic device, dental floss, a dental suture, or a denture.

18. A method for treating or preventing a bacterial infection in an oral cavity of a subject in need thereof comprising using the article of claim 1.

19. A method for treating or preventing a periodontal disease in a subject in need thereof comprising using to the article of claim 1.

20. A method for preventing or reducing the formation of biofilm or dental plaque in a subject in need thereof comprising using the article of claim 1.
